(19) United States
(12) Patent Application Publication
(10) Pub. No.: US 2026/0115394 A1
(43) Pub. Date: Apr. 30, 2026

(54) PHOTOLUMINESCENT SLEEVE DEVICE

(71) Applicant: Prisca Harrison, Chehalis, WA (US)

(72) Inventor: Prisca Harrison, Chehalis, WA (US)

(21) Appl. No.: 18/925,242

(22) Filed: Oct. 24, 2024

(51) Int. Cl. A61M 16/00; A61M 16/06

(52) U.S. Cl. CPC A61M 16/0003; A61M 16/0666; A61M 2205/587

(57) ABSTRACT

A photoluminescent sleeve device for enabling a nasal canula to be observed in a darkened environment includes a nasal cannula and a sleeve that has an entry extending along a full length of the sleeve. The entry enables a tube of the nasal cannula to be inserted into the sleeve when the nasal cannula is being worn on a patient. The sleeve is comprised of a photoluminescent material such that the sleeve is visible in a darkened environment. In this way a caregiver that is remotely monitoring the patient is enabled to determine when the nasal cannula has become dislodged on the patient while the patient is sleeping during nighttime hours.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sleeve devices and more particularly pertains to a new sleeve device for enabling a nasal cannula to be visible in a darkened environment. The device includes a sleeve that has an entry that extends along a full length of the sleeve to enable the entry to insertably receive a tube of a nasal cannula. In this way the sleeve is oriented to extend along a length of the nasal cannula that is positioned on a patient's face. The sleeve is comprised of a photoluminescent material thereby enabling the sleeve to be visible to a caregiver that is remotely monitoring the patient while the patient is sleeping during nighttime hours.

### (2) Description of Related Art including information disclosed under 37 CFR 1.97 and 1.98

The prior art relates to sleeve devices including: a variety of illuminated medical tubing devices that each at least includes an optical element that employs electricity to emit visible light thereby enabling a medical tube to be visible in a darkened environment; a variety of medical line identification devices that each at least includes an optical fiber that emits light from a light source to enable a medical line to be visible in a darkened environment; a variety of medical line identification devices that each at least includes a light that is disposed on a medical line to enable the medical line to be visible in a darkened environment. In no instance does the prior art disclose a photoluminescent sleeve device that includes a sleeve, comprised of a photoluminescent material, which has an entry which extends along a full length of the sleeve to enable the sleeve to be positioned on a tube of a nasal cannula thereby enabling the nasal cannula to be visible to a caregiver that is remotely monitoring a patient while the patient is sleeping during nighttime hours.

### BRIEF SUMMARY OF THE INVENTION

An embodiment of the disclosure meets the needs presented above by generally comprising a nasal cannula and sleeve that has an entry extending along a full length of the sleeve. The entry enables a tube of the nasal cannula to be inserted into the sleeve when the nasal cannula is being worn on a patient. The sleeve is comprised of a photoluminescent material such that the sleeve is visible in a darkened environment. In this way a caregiver that is remotely monitoring the patient is enabled to determine when the nasal cannula has become dislodged on the patient while the patient is sleeping during nighttime hours.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective phantom view of a photoluminescent sleeve device according to an embodiment of the disclosure.

FIG. 2 is a right side view of an embodiment of the disclosure.

FIG. 3 is a perspective in-use view of an embodiment of the disclosure showing a sleeve being positioned on a tube of a nasal cannula.

FIG. 4 is a perspective in-use view of an embodiment of the disclosure showing a caregiver remotely monitoring a patient that is wearing a nasal cannula.

### DETAILED DESCRIPTION OF THE INVENTION

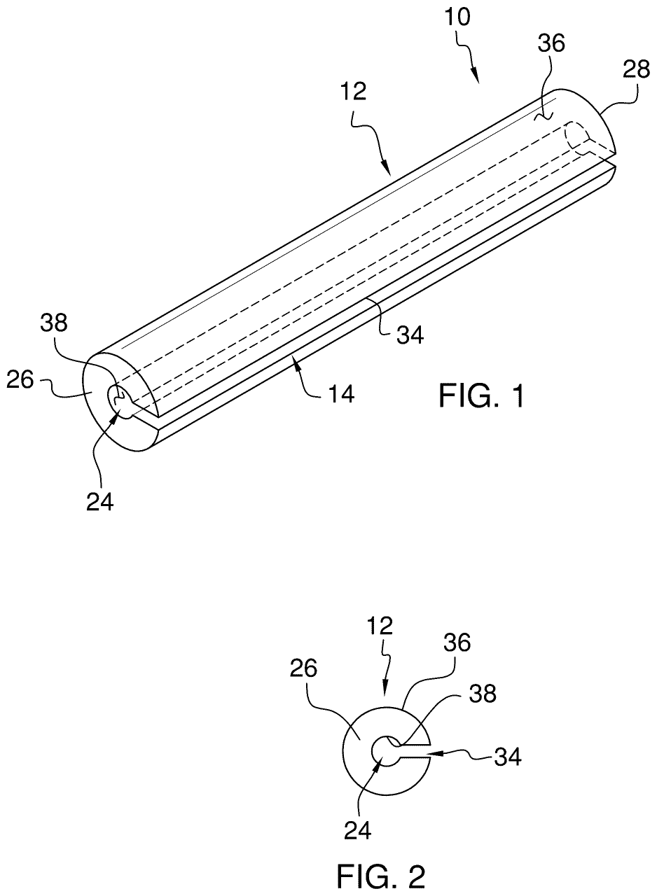

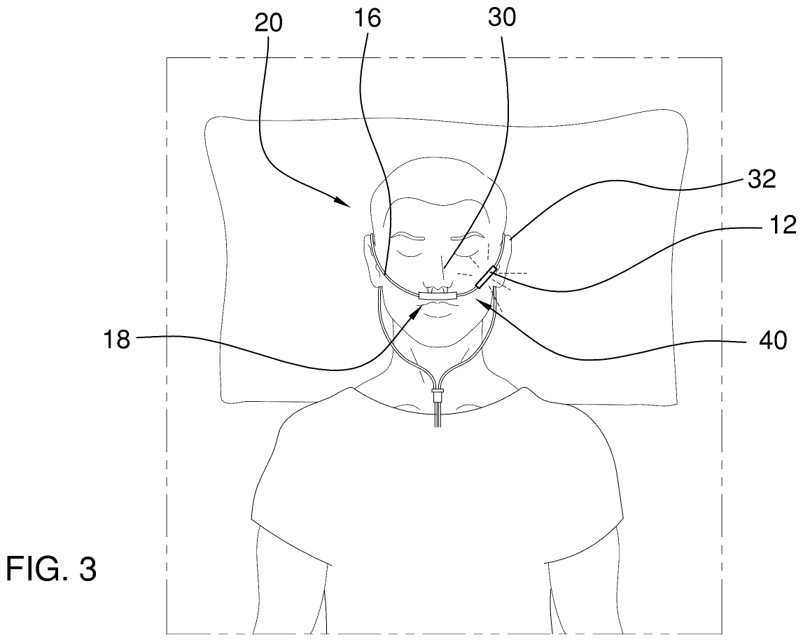

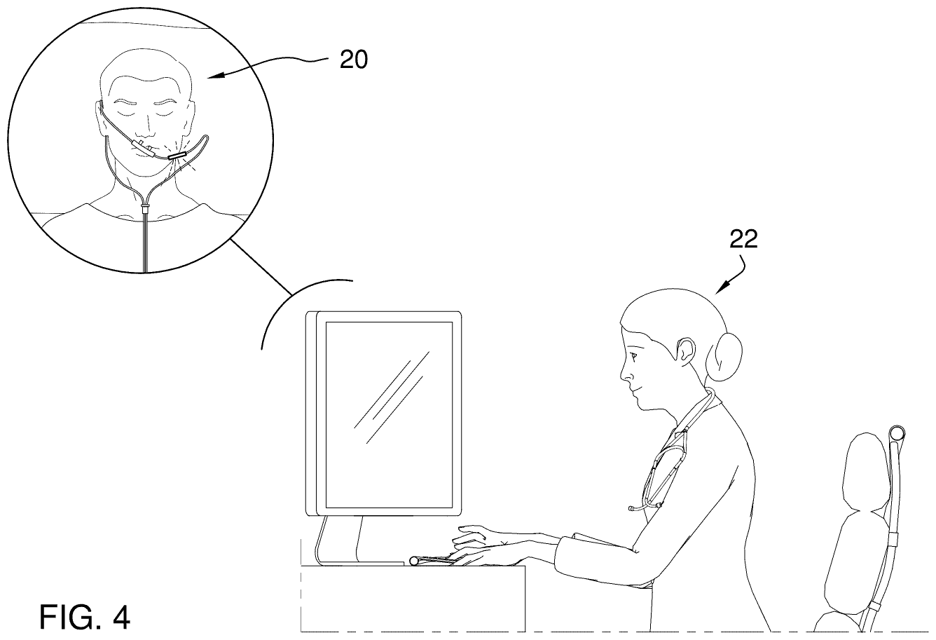

With reference now to the drawings, and in particular to FIGS. 1 through 4 thereof, a new sleeve device embodying the principles and concepts of an embodiment of the disclosure and generally designated by the reference numeral 10 will be described.

As best illustrated in FIGS. 1 through 4, the photoluminescent sleeve device 10 generally comprises a sleeve 12 which has an entry 14 extending along a full length of the sleeve 12. In this way the entry 14 enables a tube 16 of a nasal cannula 18 to be inserted into the sleeve 12 when the nasal cannula 18 is being worn on a patient 20. The patient 20 may be an elderly person with dementia or a person recovering from a surgical procedure that is expected to regain consciousness from general anesthesia or any other patient that might be prone to forcibly removing the nasal cannula 18 from their nostrils due to being confused and/or agitated. Furthermore, the nasal cannula 18 may be a nasal cannula of any conventional design that is employed in a medical environment to supply oxygen therapy to the patient 20. The sleeve 12 is comprised of a photoluminescent material, including but not being limited to silicone that is infused with either zinc sulfide or strontium aluminate, such that the sleeve 12 is visible in a darkened environment. In this way a caregiver 22 that is remotely monitoring the patient 20 can visually determine when the nasal cannula 18 has become dislodged on the patient 20 while the patient 20 is sleeping during nighttime hours. Thus, the caregiver 22 can immediately attend to the patient 20 to replace the nasal cannula 18 to avert a potentially life threatening situation for the patient 20.

The sleeve 12 has a hole 24 which extends through a first end 26 and a second end 28 of the sleeve 12 to enable the tube 16 of the nasal cannula 18 to extend through the sleeve 12. Additionally, the hole 24 is centrally positioned in the sleeve 12 to enable the tube 16 of the nasal cannula 18 to be centrally located in the sleeve 12. In this way the likelihood of the sleeve 12 falling off of the tube 16 of the nasal cannula 18 is reduced. The sleeve 12 is elongated along an axis extending between the first end 26 and the second end 28 thereby enabling the sleeve 12 to extend a distance between the patient's nose 30 and the patient's ear 32 that is equivalent to a length of the axis extending between the first end 26 and the second end 28 when the sleeve 12 is positioned on the tube 16 of the nasal cannula 18. In this way the caregiver 22 can visually confirm that the nasal cannula 18 is positioned in a preferred orientation on the patient 20.

The sleeve 12 has a slot 34 which extends between the outer surface 36 of the sleeve 12 and a bounding surface 38 of the hole 24. Furthermore, the slot 34 is elongated to extend between the first end 26 and the second end 28 such that the slot 34 defines the entry 14 in the sleeve 12. In this way the slot 34 can insertably receive the tube 16 of the nasal cannula 18 thereby enabling the tube 16 of the nasal cannula 18 to be positioned in the hole 24. Thus, the sleeve 12 can be oriented to extend along a length of the tube 16 that is sufficient to enable the sleeve 12 to be visually identified on the tube 16.

In use, the sleeve 12 is positioned on the tube 16 of the nasal cannula 18 such that the sleeve 12 is positioned on the patient's cheek 40 when the patient 20 is wearing the nasal cannula 18. In this way the sleeve 12 is visible on the patient 20 to enable the caregiver 22 to visually confirm that the nasal cannula 18 is positioned in the preferred orientation on the patient 20. Furthermore, the sleeve 12 is photoluminescent thereby enabling the caregiver 22 to see the sleeve 12 during nighttime hours. In this way the caregiver 22 can visually identify that the nasal cannula 18 is in the preferred orientation while the patient 20 is sleeping during nighttime hours. Additionally, the sleeve 12 enables the caregiver 22 to visually identify when the nasal cannula 18 has become dislodged thereby enabling the caregiver 22 to avert a potentially life-threatening situation for the patient 20.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded.

A reference to an element by the indefinite article “a” does not exclude the possibility that more than one of the element is present, unless the context clearly requires that there be only one of the elements.

I claim:

1. A photoluminescent sleeve device being positionable around a nasal cannula thereby enabling said nasal cannula to be visible to a caregiver in a darkened environment, said device comprising:
a nasal cannula having a tube and a pair of nasal ports wherein said nasal cannula is configured to be worn on a patient having said pair of nasal ports extending into nostrils of the patient and having said tube being positioned on the patient's face; and
a sleeve having an entry extending along a full length of said sleeve, said entry insertably receiving said tube of said nasal cannula into when said nasal cannula is being worn on the patient, said sleeve being comprised of a photoluminescent material wherein said sleeve is configured to be visible in a darkened environment thereby enabling a caregiver that is remotely monitoring the patient to determine when said nasal cannula has become dislodged on the patient while the patient is sleeping during nighttime hours.

2. The photoluminescent sleeve device according to claim 1, wherein:
said sleeve has a hole extending through a first end and a second end of said sleeve; and
said hole enabling said tube of said nasal cannula to extend through said sleeve.

3. The photoluminescent sleeve device according to claim 2, wherein said hole is centrally positioned in said sleeve thereby enabling said tube of said nasal cannula to be centrally located in said sleeve thereby reducing the likelihood that said sleeve will fall off of said tube of said nasal cannula.

4. The photoluminescent sleeve device according to claim 2, wherein said sleeve is elongated along an axis extending between said first end and said second end wherein said sleeve is configured to extend a distance between the patient's nose and the patient's ear that is equivalent to a length of said axis extending between said first end and said second end when said sleeve is positioned on said tube of said nasal cannula thereby enabling the caregiver to visually confirm that said nasal cannula is positioned in a preferred orientation on the patient.

5. The photoluminescent sleeve device according to claim 2, wherein said sleeve has a slot extending between said outer surface of said sleeve and a bounding surface of said hole.

6. The photoluminescent sleeve device according to claim 5, wherein said slot is elongated to extend between said first end and said second end such that said slot defines said entry in said sleeve thereby enabling said slot to insertably receive said tube of said nasal cannula thereby enabling said tube of said nasal cannula to be positioned in said hole.

7. A photoluminescent sleeve device being positionable around a nasal cannula thereby enabling said nasal cannula to be visible to a caregiver in a darkened environment, said device comprising:
a nasal cannula having a tube and a pair of nasal ports wherein said nasal cannula is configured to be worn on a patient having said pair of nasal ports extending into nostrils of the patient and having said tube being positioned on the patient's face;
a sleeve having an entry extending along a full length of said sleeve, said tube of said nasal cannular being insertable into said entry when said nasal cannula is being worn on the patient, said sleeve being comprised of a photoluminescent material wherein said sleeve is configured to be visible in a darkened environment thereby enabling a caregiver that is remotely monitoring the patient to determine when said nasal cannula has become dislodged on the patient while the patient is sleeping during nighttime hours, said sleeve having a hole extending through a first end and a second end of said sleeve, said hole enabling said tube of said nasal cannula to extend through said sleeve, said hole being centrally positioned in said sleeve thereby enabling said tube of said nasal cannula to be centrally located in said sleeve thereby reducing the likelihood that said sleeve will fall off of said tube of said nasal cannula, said sleeve being elongated along an axis extending between said first end and said second end wherein said sleeve is configured to extend a distance between the patient's nose and the patient's ear that is equivalent to a length of said axis extending between said first end and said second end when said sleeve is positioned on said tube of said nasal cannula thereby enabling the caregiver to visually confirm that said nasal cannula is positioned in a preferred orientation on the patient, said sleeve having a slot extending between said outer surface of said sleeve and a bounding surface of said hole, said slot being elongated to extend between said first end and said second end such that said slot defines said entry in said sleeve thereby enabling said slot to insertably receive said tube of said nasal cannula thereby enabling said tube of said nasal cannula to be positioned in said hole.

8. A method of enabling a nasal cannula to be visible in a darkened environment to a caregiver that is remotely monitoring a patient who is wearing said nasal cannula, the steps of the method comprising:
inserting a tube of a nasal cannula into a slot which extends through an outer surface of a sleeve; and
positioning said tube of said nasal cannula in a hole which extends between a first end and a second end of said sleeve wherein said sleeve is configured to be positioned on a patient's face when said nasal cannula is being worn by the patient, said sleeve being comprised of a photoluminescent material wherein said sleeve is configured to be visible in a darkened environment thereby enabling a caregiver that is remotely monitoring the patient to visually confirm that said nasal cannula is positioned in a preferred orientation on the patient.
